(19) United States
(12) Patent Application Publication
(10) Pub. No.: US 2025/0049602 A1
(43) Pub. Date: Feb. 13, 2025

(54) ELECTRIC TASTE DEVICE

(71) Applicant: Nanae Terazaki, Portland, OR (US)

(72) Inventor: Nanae Terazaki, Portland, OR (US)

(21) Appl. No.: 18/448,835

(22) Filed: Aug. 11, 2023

(51) Int. Cl. A61F 5/00; A61N 1/36

(52) U.S. Cl. CPC A61F 5/0006; A61N 1/36014; A61M 2021/0072

(57) ABSTRACT

An electric band device having a band and an attachment portion assembly to enhance salt taste is disclosed. The electric band device includes a band portion, which can be wrapped around a bowl. The attachment portion assembly can be attached to the band portion and provides electric power supply. An electric straw device and an electric cutlery device are also disclosed. The user can use their own bowls, straws, cutlery of variety of choices to get the benefits of electric taste.

### BACKGROUND

### Technical Field

The disclosure is related to an electric taste device, and particularly to an electric band device having a band and an attachment to enhance salt taste.

### Description of Related Art

It is widely recommended by health professionals to reduce salt intake. Reducing salt intake is important for longevity and particularly important to people who have diabetes or high-blood pressure issues. There are devices such as electric spoons, electric folks, or electric bowls to enhance salt taste by utilizing electric taste. In these devices, electric elements to enhance salt taste are integrated with spoons, folks, or bowls. Therefore, the design or size of these electric spoons, electric forks or electric bowls are limited and it's not possible to use the user's own spoons, forks, or bowls.

### SUMMARY

The disclosure provides an electric band device having a band and an attachment portion assembly to enhance salt taste. The electric band device includes a band portion, which can be wrapped around a bowl. An attachment portion assembly can be attached to the band portion and provides electric power supply. Because the electric band device and the bowl are independent, a user can use their own bowl, to which the electric band device can be attached to get electric taste. Therefore, the user can use their own bowls of choice to get the benefits of electric taste. Is addition, the user can use disposable containers of ready-to-eat foods as well as ceramic ware or porcelain such as a bowl to get the benefits of electric taste by wrapping the electric band device.

The disclosure also provides an electric straw device having a holder body and a straw holder. The electric straw device is configured to insert a straw. Because the electric straw device and the straw are independent, a user can use their own straw. Therefore, the user can use their own straws of choice to get the benefits of electric taste. Is addition, the user can use disposable straws as well as reusable straws.

The disclosure also provides an electric cutlery device having a space configured to insert cutlery. Because the electric cutlery device and the cutlery are independent, a user can use their own cutlery. Therefore, the user can use their own cutlery of choice to get the benefits of electric taste.

In order to make the aforementioned features and advantages of the disclosure more comprehensible, embodiments accompanying figures are described in detail below.

### BRIEF DESCRIPTION OF THE DRAWINGS

FIG. 1 is a schematic front view of an electric band device according to an embodiment of the disclosure.

FIG. 2 is a schematic bottom view of an electric band device according to an embodiment of the disclosure.

FIG. 3 is a panorama diagram of an electric band device attached to a bowl according to an embodiment of the disclosure.

FIG. 4 is a schematic top view of an electric band device when attached to a bowl according to an embodiment of the disclosure.

FIG. 5 is a diagram of an electric band device according to an embodiment of the disclosure.

FIG. 6 is a panorama diagram of an electric straw device according to an embodiment of the disclosure.

FIG. 7 is schematic front view of an electric straw device according to an embodiment of the disclosure.

FIG. 8 is a cross-sectional view of an electric straw device taken along the A-A line in FIG. 9 according to an embodiment of the disclosure.

FIG. 9 is schematic top view of an electric straw device according to an embodiment of the disclosure.

FIG. 10 is schematic top view of a modified example of an electric straw device according to an embodiment of the disclosure.

FIG. 11 is a diagram of an electric straw device according to an embodiment of the disclosure.

FIG. 12 is schematic top view of a modified example of an electric straw device according to an embodiment of the disclosure.

FIG. 13 is a panorama diagram of an electric cutlery device according to an embodiment of the disclosure.

FIG. 14 is schematic front view of an electric cutlery device according to an embodiment of the disclosure.

FIG. 15 is schematic top view of an electric cutlery device according to an embodiment of the disclosure.

FIG. 16 is schematic side view of an electric cutlery device according to an embodiment of the disclosure.

FIG. 17 shows an inner surface of a modified example of an electric cutlery device according to an embodiment of the disclosure.

FIG. 18 shows an inner surface of another modified example of an electric cutlery device according to an embodiment of the disclosure.

FIG. 19 shows an inner surface of another modified example of an electric cutlery device according to an embodiment of the disclosure.

FIG. 20 is a diagram of an electric cutlery device according to an embodiment of the disclosure.

### DETAILED DESCRIPTION OF THE INVENTION

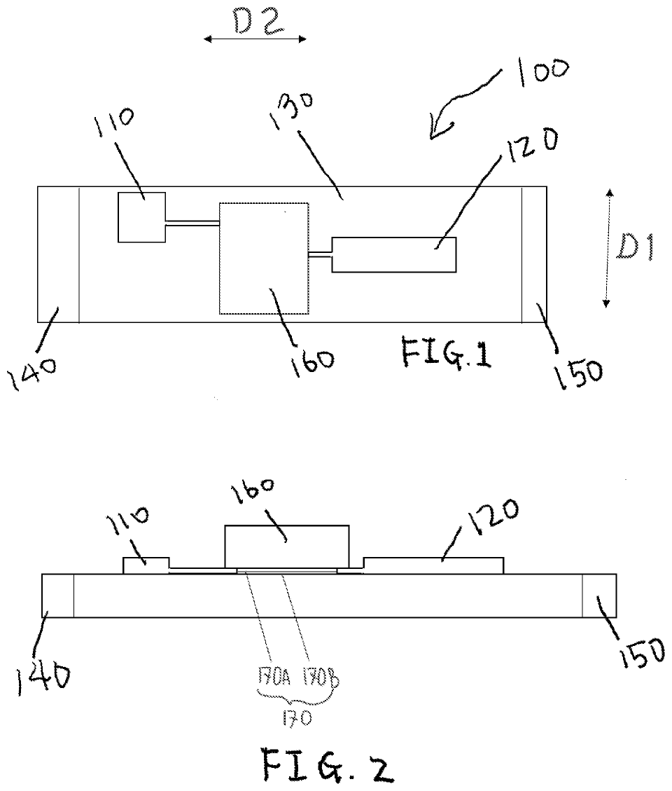

Referring to FIG. 1 and FIG. 2, in the embodiment, an electric band device 100 includes a mouth contact portion 110, a hand contact portion 120. The mouth contact portion 110 and the hand contact portion 120 are connected to an attachment portion assembly 165 when the attachment portion assembly 165 is attached to a band portion 130. The attachment portion assembly 165 includes electric elements. The mouth contact portion 110 and the hand contact portion 120 are conductive and are respectively electrically connected to the power supply 200 in the attachment portion assembly 165. The mouth contact portion 110 and the hand contact portion 120 are electrically insulated from each other. The mouth contact portion 110 and the hand contact portion 120 can be made of metal, conductive plastic, conductive resin, conductive fabric, conductive ink etc.

The mouth contact portion 110 and the hand contact portion 120 are located on or embedded in a band portion 130. The band portion 130 is electrically insulating and can be elastic and stretchable. The band portion 130 has a width direction D1 and longitudinal direction D2, where the longitudinal direction D2 is longer than the width direction D1. The band portion 130 includes a first connection portion 140 and a second connection portion 150 at both ends of the electric band device 100 when viewed in the longitudinal direction D2.

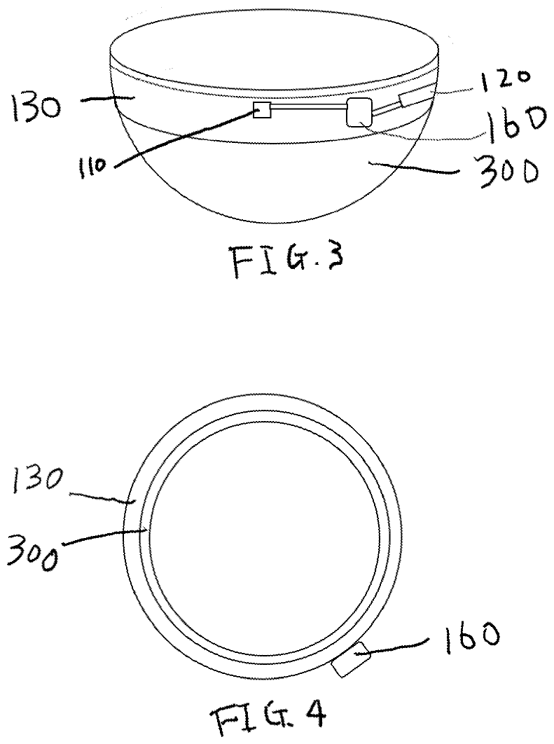

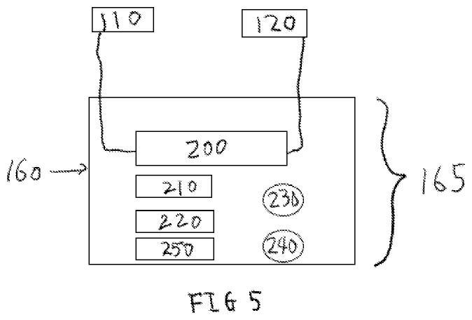

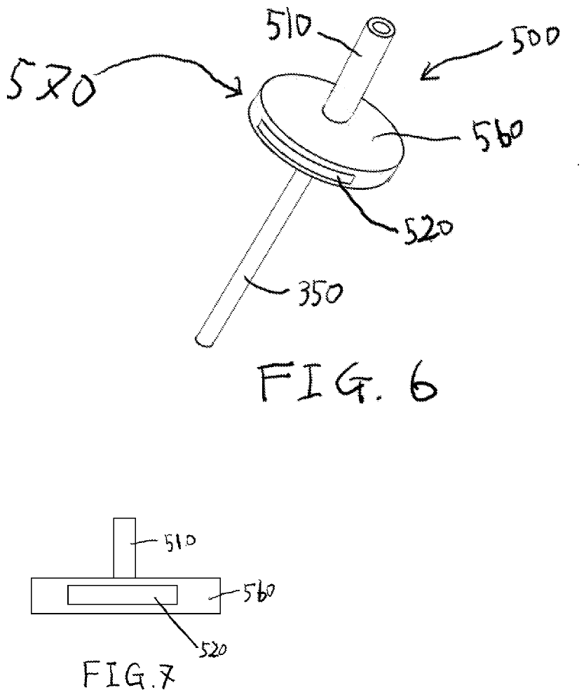

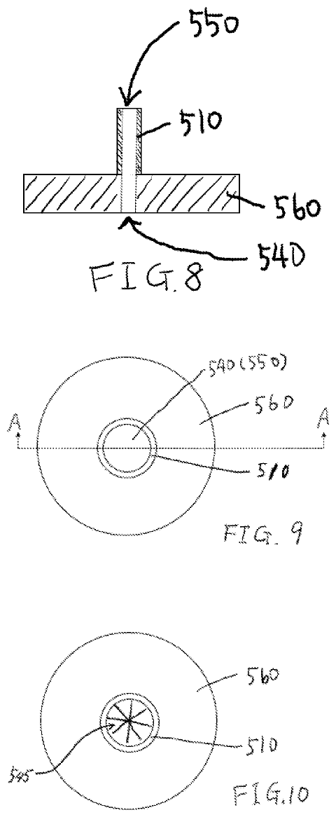

The electric band device 100 can be wrapped around a bowl 300 near the top opening of the bowl 300 as shown in FIG. 3 and FIG. 4. If desired, the electric band device 100 can be wrapped around a cup, any other tableware, dinnerware, or a disposable container of ready-to-eat foods etc. At least a part of the band portion 130 is elastic and stretchable and therefore, the electric band device 100 can be easily wrapped around the bowl 300. Both ends of the electric band device 100 can be connected to each other by the first connection portion 140 and the second connection portion 150. (see FIG. 3 and FIG. 4). The first connection portion 140 and the second connection portion 150 can be a pair of magnets, snaps, fasteners, hooks etc. Also, when the first connection portion 140 and the second connection portion 150 are connected, they can be partially overlapping, completely overlapping or not overlapping.

The attachment portion assembly 165 can be attached to the band device 100 by a third connection portion 170. The third connection portion 170 can be a pair of two connection portions, i.e., an attachment-side connection portion 170A and a band-side connection portion 170B, which can be a pair of magnets, snaps, fasteners, or hooks etc. (FIG. 2). One of the pair of magnets, snaps, fasteners, or hooks etc. is placed on the attachment portion 160 and the other of the pair of magnets, snaps, fasteners or hooks etc. can be placed on the band portion 130. For example, the attachment-side connection portion 170A is a magnet M1 and a band-side connection portion 170B is a magnet M2, where the magnet M1 is placed on the attachment portion 160 and the magnet M2 is placed on the band portion 130. Therefore, the attachment portion assembly 165 and the band portion 130 can be attached to each other by the magnetic force. Also, the attachment portion assembly 165 can be attachable to or detachable from the band portion 130 easily.

When the user eats food or soup, or drinks beverage, the user's hand touches the hand contact portion 120 and the user's mouth touches the month contact portion 110. As described above, the mouth contact portion 110 and the hand contact portion 120 are conductive and are respectively electrically connected to the power supply 200 in the attachment portion assembly 165. As a result, an electric circuit is established through a part of the electric band device 100 and the human body and an electric current can be carried through the human body. The electric current can be small enough to be safe to the human body and within the range wherein effective or desirable electric taste can be obtained.

The attachment portion assembly 165 includes the attachment portion 160 and electric elements. The attachment portion 160 is electrically insulating and is a case having a hollow space to contain at least a powder supply 200 and optionally includes a noise reduction element 210, a circuit feedback control element 220, a current control element 250. (see FIG. 5). The powder supply 200, the noise reduction element 210, the circuit feedback control element 220, the current control element 250 may be placed in the hollow space in the attachment portion 160. The first button 230 and the second button 240 may be placed on the attachment portion 160 so that the user can powder on/off by the first button 230 and control the applied voltage by the second button 240 to get preferred strength of electric taste. Therefore, the attachment portion assembly 165 can provide desirable voltage, desirable current, desirable voltage waveform, and desirable current waveform.

In summary of the above, the electric band device 100 of the disclosure can provide the user's desired electric taste while making it possible to use the user's desired bowls 300 or containers.

A second embodiment regarding an electric straw device is explained as the following.

Referring to FIG. 6 and FIG. 7, in the embodiment, an electric straw device 500 includes a straw holder 510, a hand hold portion 520, and a holder body assembly 570 having a holder body 560. The straw holder 510 is connected to a holder body assembly 570, and a hand hold portion 520 is placed on the holder body assembly 570. The hand hold portion 520 may be placed on the side surface of the holder body assembly 570.

The holder body assembly 570 includes a holder body 560 and electric elements.

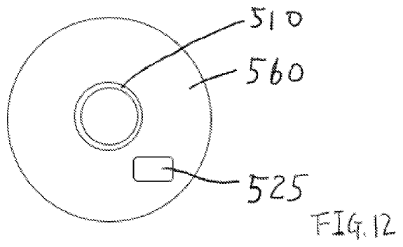

The holder body 560 is electrically insulating and is a case having a hollow space to contain electrical elements such as power supply 200, noise reduction element 210, circuit feedback control element 220, and current control element 250 etc. (see FIG. 11).

The straw holder 510 is at least partially conductive and the hand hold portion 520 is conductive. Of course, the whole portion of the straw holder 510 can be conductive also. The conductive portion of the straw holder 510 and the hand hold portion 520 are respectively electrically connected to the power supply 200 in the holder body assembly 570. The conductive portion of the straw holder 510 and the hand hold portion 520 are electrically insulated from each other. The conductive part of the straw holder 510 and the hand hold portion 520 can be made of metal, conductive plastic, conductive resin, conductive fabric, conductive ink etc.

The electric straw device 500 is configured to insert a straw 350. The holder body 560 has a first hole 540 and the straw holder 510 has a second hole 550. Both the first hole 540 and the second hole 550 are through holes and they are coaxial to each other to insert the straw 350. (see FIG. 8). The holder body 560 can be at least partially elastic particularly around the first hole 540 so that the straw 350 can be fitted and held with appropriate force such as elastic force. Also, when the holder body 560 is not elastic, an elastic ring can be inserted in the first hole 540 so that the straw 350 can be fitted and held in the first hole 540.

The diameter of the second hole 550 may be a little larger than the diameter of the first hole 540, and the straw holder 510 does not have to be elastic because the straw 350 can be fitted and held in the first hole 540 of the holder body 560.

The straw 350 can be conductive, non-conductive, or insulating and can be made of metal, bamboo, silicon, or plastic etc. The straw 350 can be inserted to the top of the straw holder 510 or middle way of the straw holder 510.

When the user drinks beverage, or eats soup, the user's hand touches the hand hold portion 520 and the user's mouth touches the straw holder 510. As described above, the straw holder 510 and the hand hold portion 520 are respectively electrically connected to the power supply 200 in the holder body assembly 570. As a result, an electric circuit is established through a part of the electric straw device 500 and the human body, and an electric current can be carried through the human body. The electric current can be small enough to be safe to the human body and within the range wherein effective or desirable electric taste can be obtained.

The holder body assembly 570 includes at least a powder supply 200 and optionally includes a noise reduction element 210, a circuit feedback control element 220, a current control element 250, a first button 230 and a second button 240. The powder supply 200, the noise reduction element 210, the circuit feedback control element 220, and the current control element 250 may be placed in the hollow space in the holder body 560. The first button 230 and the second button 240 may be placed on the holder body 560 so that the user can powder on/off by the first button 230 and control the applied voltage by the second button 240 to get preferred strength of electric taste. (see FIG. 11). Therefore, the holder body assembly 570 can provide desirable voltage, desirable current, desirable voltage waveform, and desirable current waveform.

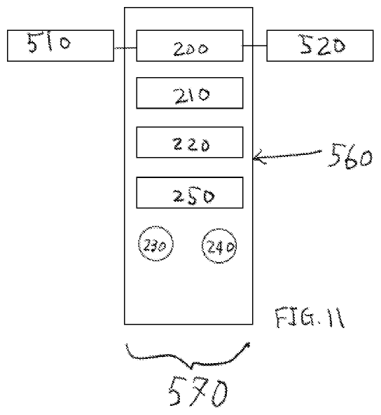

FIG. 10 shows a top view of another example of the electric straw device. In this example, an elastic sheet-like material is placed in the first hole 540, and the elastic sheet-like material has plurality of slits 545, which are radially cut. Therefore, the straw 350 can be fitted and held more stably.

FIG. 12 shows a top view of another example of the electric straw device. In this example, a top hand hold portion 525 is placed on the top surface of the holder body 560 instead of placing the hand hold portion 520 on the side surface of the holder body 560.

The shape of the holder body 560 can be circular cylinder as shown in FIG. 6, and also it can be ball shape, sphere, rugby ball shape, cuboid shape, random shape, and any other possible shapes.

In summary of the above, the electric straw device 500 of the disclosure can provide the user's desired electric taste while making it possible to use the user's desired straws 350.

A third embodiment regarding an electric cutlery device is explained as the following.

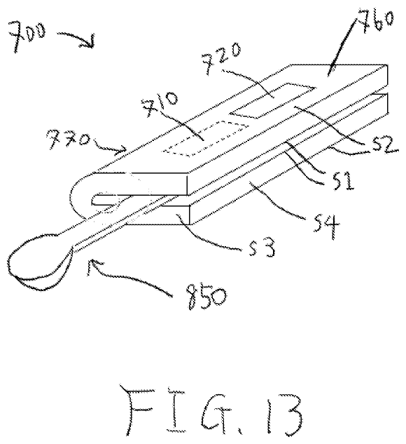

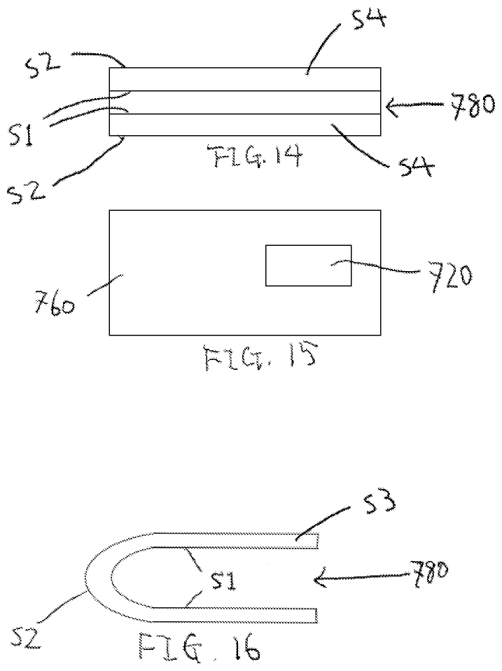

Referring to FIG. 13-FIG. 16, in the embodiment, an electric cutlery device 700 includes a cutlery contact portion 710, a handle contact portion 720, and a handle body assembly 770 having a handle body 760. The handle body 760 has an inner surface S1, an outer surface S2, side surfaces S3, and front surfaces S4. The inner surface S1 and the outer surface S2 may be curved and make a U-shape as shown in FIG. 13-FIG. 16. The surface area of an inner surface S1 is larger than the side surfaces S3; also, the surface area of an inner surface S1 is larger than the front surfaces S4; the surface area of the outer surface S2 is larger than the surface area of the inner surface S1. The handle body 760 is configured to insert cutlery such as spoons, folks, and knives. For example, the handle body 760 may have an insertion space 780 having a U-shape to provide a space to insert the cutlery 850. The handle body 760 may be at least partially elastic particularly near the inner surface S1 so that the cutlery 850 can be fitted and held with appropriate force such as elastic force. Also, when the handle body 760 is not elastic, an elastic material can be placed on the inner surface S1 so that the cutlery 850 can be fitted and held in the insertion space 780.

The cutlery contact portion 710 is placed on the inner surface S1 of a handle body 760, and a handle contact portion 720 is placed on the outer surface S2 of the handle body 760. The handle body 760 is electrically insulating and is a case having a hollow space to contain electrical elements such as power supply 200, noise reduction element 210, circuit feedback control element 220, and current control element 250 etc.

The cutlery contact portion 710 and the handle contact portion 720 are conductive. The cutlery contact portion 710 and the handle contact portion 720 are respectively electrically connected to the power supply 200 in the handle body assembly 770. The cutlery contact portion 710 and the handle contact portion 720 are electrically insulated from each other. The cutlery contact portion 710 and the handle contact portion 720 can be made of metal, conductive plastic, conductive resin, conductive fabric, conductive ink etc.

The cutlery 850 should be at least partially conductive to be able to be electrically connected to the cutlery contact portion 710. More particularly, a portion of the cutlery 850 that contacts the user's mouth should be electrically connected to the cutlery contact portion 710 through the handle portion of the cutlery 850.

When the user eats foods or soups, or drinks beverage with the cutlery 850, the user's hand touches the handle contact portion 720 and the user's mouth touches the cutlery 850, which is electrically connected to the cutlery contact portion 710. As described above, cutlery contact portion 710 and the handle contact portion 720 are respectively electrically connected to the power supply 200 in the handle body assembly 770. As a result, an electric circuit is established through a part of the electric cutlery device 700 and the human body and an electric current can be carried through the human body. The electric current can be small enough to be safe to the human body and within the range wherein effective or desirable electric taste can be obtained.

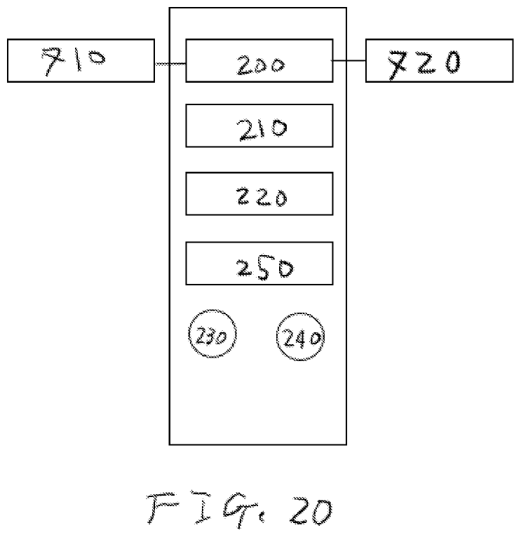

The handle body assembly 770 includes at least a powder supply 200 and optionally includes a noise reduction element 210, a circuit feedback control element 220, a current control element 250, a first button 230 and a second button 240. The powder supply 200, the noise reduction element 210, the circuit feedback control element 220, and the current control element 250 may be placed in the hollow space in the handle body 760. The first button 230 and the second button 240 may be placed on the outer surface S2 of the handle body 760 so that the user can powder on/off by the first button 230 and control the applied voltage by the second button 240 to get preferred strength of electric taste. (see FIG. 20). Therefore, the handle body assembly 770 can provide desirable voltage, desirable current, desirable voltage waveform, and desirable current waveform.

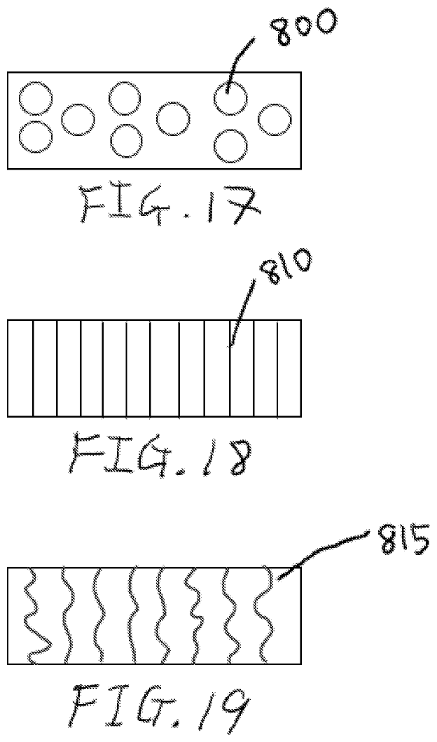

FIG. 17 shows the inner surface S1 of the handle body 760 of another example of the electric cutlery device 700. In this example, the inner surface S1 of the handle body 760 has plurality of bumps 800. Therefore, the cutlery 850 can be easily inserted and/or removed/taken out from the electric cutlery device 700, and also fitted and held more stably in the electric cutlery device 700.

FIG. 18 shows the inner surface S1 of the handle body 760 of another example of the electric cutlery device 700. In this example, the inner surface S1 of the handle body 760 has plurality of straight protrusions 810. Therefore, the cutlery 850 can be easily inserted and/or removed/taken out from the electric cutlery device 700, and also fitted and held more stably in the electric cutlery device 700.

FIG. 19 shows the inner surface S1 of the handle body 760 of another example of the electric cutlery device 700. In this example, the inner surface S1 of the handle body 760 has plurality of curved protrusions 815. Therefore, the cutlery 850 can be easily inserted and/or removed/taken out from the electric cutlery device 700, and also fitted and held more stably in the electric cutlery device 700.

In summary of the above, the electric cutlery device 700 of the disclosure can provide the user's desired electric taste while making it possible to use the user's desired cutlery 850.

Although the disclosure has been disclosed by the above embodiments, the embodiments are not intended to limit the disclosure. It will be apparent to those skilled in the art that various modifications and variations can be made to the structure of the disclosure without departing from the scope or spirit of the disclosure. Therefore, the protecting range of the disclosure falls in the appended claims.

What is claimed is:

1. An electronic band device, comprising:
a band portion, the band portion being non-conductive;
an attachment portion configured to be attached to the band portion;
a mouth contact portion electrically connected to a power supply in the attachment portion;
a hand contact portion electrically connected to the power supply and electrically insulated from the mouth contact portion;
a first connection portion on the band portion;
a second connection portion on the band portion, configured to be connected to the first connection portion; and
a pair of third connection portions including an attachment-side connection portion located on the attachment portion, and a band-side connection portion located on the band portion, wherein
the attachment portion is configured to be connected to the band portion by the pair of third connection portions.

2. The electronic band device according to claim 1, wherein the band portion is elastic.

3. An electronic straw device, comprising:
a holder body having a first hole, the holder body being non-conductive;
a straw holder having a second hole, the straw holder electrically connected to a power supply in the holder body; and
a hand hold portion electrically connected to the power supply and electrically insulated from the straw holder,
wherein the first hole and the second hole are coaxial through holes configured to insert a straw.

4. The electronic straw device according to claim 3, wherein the holder body is elastic.

5. An electronic cutlery device, comprising:
a handle body configured to insert cutlery, the handle body being non-conductive;
a cutlery contact portion on the handle body, electrically connected to a power supply in the handle body; and
a handle contact portion on the handle body, electrically connected to the power supply and electrically insulated from the cutlery contact portion.

6. The electronic cutlery device according to claim 5, wherein the handle body is elastic.
